(19) United States
(12) Patent Application Publication
(10) Pub. No.: US 2020/0354686 A1
(43) Pub. Date: Nov. 12, 2020

(54) ARTIFICIAL TISSUE COMPRISING ARTIFICIAL PAPILLARY LAYER, ARTIFICIAL SKIN AND METHOD OF MANUFACTURING SAME

(71) Applicant: POSTECH Research and Business Development Foundation, Pohang-si (KR)

(72) Inventors: Sungjune JUNG, Pohang-si (KR); Ju An PARK, Ulsan (KR); Hwa-Rim LEE, Cheongju-si (KR)

(21) Appl. No.: 16/861,446

(22) Filed: Apr. 29, 2020

(30) Foreign Application Priority Data
May 8, 2019 (KR) 10-2019-0053748

(51) Int. Cl. C12N 5/071

(52) U.S. Cl. CPC C12N 5/0698; C12N 5/0626; C12N 2535/10; C12N 2533/70; C12N 2533/50

(57) ABSTRACT

Disclosed are an artificial tissue including an artificial papillary layer, an artificial skin and a method of manufacturing the same. The artificial tissue includes an extracellular matrix layer including a first extracellular matrix and an artificial papillary layer formed on the extracellular matrix layer and including a papilla or papillae, each papilla including a cell and a second extracellular matrix, thereby overcoming the structural limitation of the flat dermis of existing artificial tissue, exhibiting excellent ability to mimic human physiology, and enabling application thereof to the production of various tissues and organs having a fine uneven structure in the human body.

### CROSS REFERENCE TO RELATED APPLICATION

The present application claims priority based on Korean Patent Application No. 10-2019-0053748, filed on May 8, 2019, the entire content of which is incorporated herein for all purposes by this reference.

### BACKGROUND OF THE INVENTION

### 1. Technical Field

The present invention relates to an artificial tissue including an artificial papillary layer, an artificial skin, and a method of manufacturing the same.

### 2. Description of the Related Art

In the field of tissue engineering and regenerative medicine, there is urgent need to replace technologies dependent upon animal experimentation or to fabricate implantable artificial tissues by manufacturing artificial tissues that closely mimic human physiology by mimicking micro-environments inside the human body using cells and cell-friendly biomaterials.

Many research groups have already worked on manufacturing artificial tissues that mimic the human body using biomaterials and multiple cells, and the resultant tissues may be used for implantation, cosmetics, and drug testing. In conventional techniques, artificial tissues having a layer structure have been manufactured by simply stacking materials and cells, but have a limitation in that they cannot mimic the interlayer microstructure of real human tissue.

In the case of human skin, a dermo-epidermal junction is present between the dermal layer and the epidermal layer, and the uneven structure under the epidermis due to the protruding dermal papillary layer of the dermo-epidermal junction also affects the function of actual skin tissue.

Therefore, there is the need for a method of manufacturing a tissue capable of more closely simulating human skin functions by mimicking the uneven structure of the dermo-epidermal junction.

### SUMMARY OF THE INVENTION

Accordingly, an objective of the present invention is to provide an artificial tissue capable of overcoming the structural limitation of the flat dermal layer of existing artificial tissue.

Another objective of the present invention is to provide a method of manufacturing an artificial tissue including an artificial papillary layer by performing cell arrangement, rather than a general process of performing surface patterning using a hydrogel alone.

Still another objective of the present invention is to provide an artificial skin capable of more closely simulating the function of real human skin by manufacturing the artificial skin including the artificial tissue and a method of manufacturing the same.

An aspect of the present invention provides an artificial tissue, including an extracellular matrix layer including a first extracellular matrix and an artificial papillary layer formed on the extracellular matrix layer and including a papilla or papillae, each papilla including a cell and a second extracellular matrix.

Also, the second extracellular matrix may be induced by the cell and contracted.

Also, the cell may include an extracellular matrix receptor, the second extracellular matrix may include at least one selected from the group consisting of a protein and a polysaccharide, and an adhesion adhering the extracellular matrix receptor to at least one selected from the group consisting of a protein and a polysaccharide may be formed. Also, the adhesion may be formed by at least one selected from the group consisting of van der Waals attraction, electrostatic attraction, ionic bonding, hydrogen bonding, and covalent bonding.

Also, the extracellular matrix receptor may include at least one selected from the group consisting of integrin, discoidin domain receptor, glycoprotein VI, immunoglobulin-like cell adhesion molecule, selectin, syndecan, dystrophin glycoprotein complex, and hyaladherin.

Also, the cell may include at least one selected from the group consisting of a fibroblast, a myoblast, a nerve cell, and a vascular endothelial cell.

Also, the fibroblast may include at least one selected from the group consisting of a mammalian fibroblast, an avian fibroblast, a reptile fibroblast, an amphibian fibroblast, and a fish fibroblast.

Also, the artificial papillary layer may have a shape corresponding to a pattern.

Also, the pattern may include at least one selected from the group consisting of a straight-line shape, a curved shape, a polygonal shape, a circular shape, an oval shape, an arc shape, a fan shape and combinations thereof.

Also, each of the extracellular matrix layer and the artificial papillary layer may be a hydrogel.

Also, each of the first extracellular matrix and the second extracellular matrix may independently include at least one selected from the group consisting of collagen, gelatin, nanocellulose, fibrinogen, hyaluronic acid, gelatin methacrylate (GelMA), and elastin.

Another aspect of the present invention provides an artificial skin, including an extracellular matrix layer including a first extracellular matrix, an artificial papillary layer formed on the extracellular matrix layer and including a papilla or papillae, and an artificial epidermal layer formed on the artificial papillary layer and including a keratinocyte, each papilla including a cell and a second extracellular matrix.

Also, the artificial epidermal layer may further include at least one selected from the group consisting of an epidermal cell and a melanocyte.

Still another aspect of the present invention provides a method of manufacturing an artificial tissue, including (a) preparing a mixed solution including a first extracellular matrix in a vessel, (b) forming a bioink pattern by discharging bioink including a cell and a second extracellular matrix on the mixed solution, and (c) culturing the mixed solution and the bioink pattern.

Also, in step (c), each of the first extracellular matrix and the second extracellular matrix may independently be cured by at least one selected from the group consisting of van der Waals attraction, hydrogen bonding, ionic bonding, and covalent bonding.

Also, the curing may occur due to a decrease in hydrogen bonding with a water molecule independently of each of the first extracellular matrix and the second extracellular matrix owing to an elevation in temperature, and due to an increase in self-aggregation owing to van der Waals attraction.

Also, the artificial tissue may include an extracellular matrix layer including the first extracellular matrix, and an artificial papillary layer formed on the extracellular matrix layer and including a papilla or papillae, each papilla including the cell and the second extracellular matrix.

Also, the cell may include an extracellular matrix receptor, the second extracellular matrix may include at least one selected from the group consisting of a protein and a polysaccharide, and the papilla may be formed to protrude by contracting and compacting all or part of the second extracellular matrix due to formation of an adhesion adhering the extracellular matrix receptor to at least one selected from the group consisting of a protein and a polysaccharide. Also, the bioink pattern may be formed through any one process selected from the group consisting of micro-extrusion printing, inkjet printing, laser printing, valve-type printing, spray printing, micro-stamping, and masking.

Yet another aspect of the present invention provides a method of manufacturing an artificial skin, including (a′) preparing a mixed solution including a first extracellular matrix in a vessel, (b′) forming a bioink pattern by discharging bioink including a cell and a second extracellular matrix on the mixed solution, (c′) culturing the mixed solution and the bioink pattern, and (d′) printing and culturing a keratinocyte solution including a keratinocyte on the mixed solution and the bioink pattern, which are cultured.

The artificial tissue according to the present invention includes an artificial papillary layer including a papilla or papillae, thereby overcoming the structural limitation of the flat epidermis of existing artificial tissue.

In addition, the method of manufacturing the artificial tissue according to the present invention is capable of manufacturing an artificial tissue having excellent ability to mimic human physiology using artificial arrangement of cells rather than a general process of forming a surface pattern using a hydrogel alone, and moreover, can be utilized to produce all kinds of tissues and organs having a fine uneven structure in the human body.

In addition, the present invention is capable of applying various patterns when forming the artificial papillary layer, thus inducing an artificial papillary layer in various shapes related to the patterns, thereby providing artificial skin with a controllable state or degree of aging.

### BRIEF DESCRIPTION OF DRAWINGS

Since these drawings are for reference in describing exemplary embodiments of the present invention, the technical spirit of the present invention should not be construed as being limited to the accompanying drawings, in which:

FIG. 1 schematically shows a process of manufacturing an artificial tissue according to an embodiment of the present invention;

FIG. 2 is optical microscope images showing an artificial papillary layer formed according to a bioink pattern in the process of manufacturing an artificial tissue according to an embodiment of the present invention;

FIG. 3 shows a state in which a keratinocyte solution including keratinocytes is printed and cultured on the cultured mixed solution and bioink pattern in order to manufacture an artificial skin according to an embodiment of the present invention;

FIG. 4 is an optical microscope image showing the cross-section of the artificial skin manufactured in Example 4;

FIG. 5 shows images obtained by scanning the artificial papillary layer of the artificial tissue manufactured in Examples 1 to 3 using a 3D confocal microscope; and

FIG. 6 schematically shows the papilla size of the artificial papillary layer in the artificial tissue manufactured in Examples 1 to 3 and Comparative Examples 1 and 2.

### DESCRIPTION OF SPECIFIC EMBODIMENTS

Hereinafter, exemplary embodiments of the present invention are described in detail with reference to the appended drawings so as to be easily performed by a person having ordinary skill in the art.

However, the following description does not limit the present invention to specific embodiments, and in the description of the present invention, detailed descriptions of related known techniques incorporated herein will be omitted when the same may make the gist of the present invention unclear.

The terms herein are used to explain specific embodiments, and are not intended to limit the present invention. Unless otherwise stated, a singular expression includes a plural expression. In the present application, the terms “comprise”, “include” or “have” are used to designate the presence of features, numbers, steps, operations, elements, parts, or combinations thereof described in the specification, and should be understood as not excluding the presence or additional possible presence of one or more different features, numbers, steps, operations, elements, parts, or combinations thereof.

As used herein, the terms “first”, “second”, etc. may be used to describe various elements, but these elements are not to be limited by these terms. These terms are only used to distinguish one element from another. For example, a first element may be termed a second element, and similarly, a second element may be termed a first element, without departing from the scope of the present invention.

Further, it will be understood that when an element is referred to as being “formed” or “stacked” on another element, it can be formed or stacked so as to be directly attached to all surfaces or to one surface of the other element, or intervening elements may be present therebetween.

Hereinafter, a detailed description will be given of an artificial tissue including an artificial papillary layer, an artificial skin and a method of manufacturing the same according to the present invention, which is set forth to illustrate but is not to be construed as limiting the present invention, and the present invention is defined only by the accompanying claims.

The present invention pertains to an artificial tissue, including an extracellular matrix layer including a first extracellular matrix and an artificial papillary layer formed on the extracellular matrix layer and including a papilla or papillae, each papilla including a cell and a second extracellular matrix.

Also, the second extracellular matrix may be induced by the cell and contracted.

Also, the cell may include an extracellular matrix receptor, the second extracellular matrix may include at least one selected from the group consisting of a protein and a polysaccharide, and an adhesion adhering the extracellular matrix receptor to at least one selected from the group consisting of a protein and a polysaccharide may be formed.

The papilla or papillae may be formed by contracting the second extracellular matrix due to traction force of the cell caused by the extracellular matrix receptor in the adhesion.

Also, the adhesion may be formed by at least one selected from the group consisting of van der Waals attraction, electrostatic attraction, ionic bonding, hydrogen bonding, and covalent bonding.

Also, the extracellular matrix receptor may include at least one selected from the group consisting of integrin, discoidin domain receptor, glycoprotein VI, immunoglobulin-like cell adhesion molecule, selectin, syndecan, dystrophin glycoprotein complex, and hyaladherin.

Also, the cell may include at least one selected from the group consisting of a fibroblast, a myoblast, a nerve cell, and a vascular endothelial cell, and preferably includes a fibroblast.

Also, the fibroblast may include at least one selected from the group consisting of a mammalian fibroblast, an avian fibroblast, a reptile fibroblast, an amphibian fibroblast, and a fish fibroblast, and preferably includes a mammalian fibroblast. Most preferably, a human dermal fibroblast is used.

Also, the artificial papillary layer may have a shape corresponding to a pattern.

Also, the pattern may include at least one selected from the group consisting of a straight-line shape, a curved shape, a polygonal shape, a circular shape, an oval shape, an arc shape, a fan shape and combinations thereof.

Also, each of the extracellular matrix layer and the artificial papillary layer may be a hydrogel.

Also, each of the first extracellular matrix and the second extracellular matrix may independently include at least one selected from the group consisting of collagen, gelatin, nanocellulose, fibrinogen, hyaluronic acid, gelatin methacrylate (GelMA) and elastin, and preferably includes collagen.

The present invention pertains to an artificial epidermis, including an extracellular matrix layer including a first extracellular matrix, an artificial papillary layer formed on the extracellular matrix layer and including a papilla or papillae, and an artificial epidermal layer formed on the artificial papillary layer and including a keratinocyte, each papilla including a cell and a second extracellular matrix.

Also, the artificial epidermal layer may further include at least one selected from the group consisting of an epidermal cell and a melanocyte.

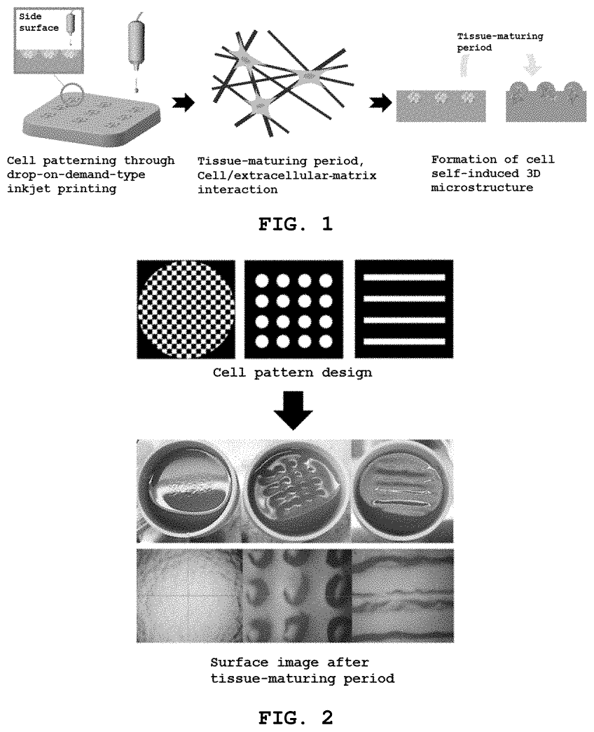

FIG. 1 schematically shows the process of manufacturing an artificial tissue according to an embodiment of the present invention.

With reference to FIG. 1, the present invention pertains to a method of manufacturing an artificial tissue, including (a) preparing a mixed solution including a first extracellular matrix in a vessel, (b) forming a bioink pattern by discharging bioink including a cell and a second extracellular matrix on the mixed solution, and (c) culturing the mixed solution and the bioink pattern.

Also, in step (c), each of the first extracellular matrix and the second extracellular matrix may independently be cured by at least one selected from the group consisting of van der Waals attraction, hydrogen bonding, ionic bonding, and covalent bonding.

Here, the curing may occur due to a decrease in hydrogen bonding with a water molecule independently of each of the first extracellular matrix and the second extracellular matrix owing to an elevation in temperature, and due to an increase in self-aggregation owing to van der Waals attraction.

Also, the artificial tissue may include an extracellular matrix layer including the first extracellular matrix and an artificial papillary layer formed on the extracellular matrix layer and including a papilla or papillae, each papilla including the cell and the second extracellular matrix.

Also, the cell may include an extracellular matrix receptor, the second extracellular matrix may include at least one selected from the group consisting of a protein and a polysaccharide, and the papilla may be formed to protrude by contracting and compacting all or part of the second extracellular matrix due to formation of an adhesion adhering the extracellular matrix receptor to at least one selected from the group consisting of a protein and a polysaccharide.

Also, the bioink pattern may be formed through any one process selected from the group consisting of micro-extrusion printing, inkjet printing, laser printing, valve-type printing, spray printing, micro-stamping, and masking.

FIG. 2 shows optical microscope images of the artificial papillary layer formed according to the bioink pattern in the process of manufacturing the artificial tissue according to an embodiment of the present invention.

With referenced to FIG. 2, in step (b), the bioink pattern may be formed in a desired shape and cell density by discharging the bioink as a droplet on the mixed solution, and the artificial papillary layer including the papilla or papillae may be obtained by forming papillae in various bioink pattern shapes.

The present invention pertains to a method of manufacturing an artificial skin, including (a′) preparing a mixed solution including a first extracellular matrix in a vessel, (b′) forming a bioink pattern by discharging bioink including a cell and a second extracellular matrix on the mixed solution, (c′) culturing the mixed solution and the bioink pattern, and (d′) printing and culturing a keratinocyte solution including a keratinocyte on the mixed solution and the bioink pattern, which are cultured.

The keratinocyte solution may further include at least one selected from the group consisting of an epidermal cell and a melanocyte.

### EXAMPLES

A better understanding of the present invention will be given through the following examples, which are merely set forth to illustrate the present invention but are not to be construed as limiting the scope of the present invention.

### Preparation Example 1: Bioink

Lyophilized Type-I pig skin extract collagen was dissolved in 0.1 v/v % acetic acid, added with a nutrient solution containing Dulbecco's Modified Eagle Medium (DMEM), Ham's F-12, and penicillin/streptomycin, uniformly mixed, added with a mixed solution of 0.05 M NaOH, NaHCO₃ and HEPES, and titrated to a neutral pH, thus preparing a mixed solution. Here, the volume ratio of NaOH to NaHCO₃ to HEPES was 8:1:1.

The mixed solution was added with human dermal fibroblasts at a concentration of 4.0×10⁶ cells/ml, thereby manufacturing bioink.

### Preparation Example 2: Bioink

Bioink was manufactured in the same manner as in Preparation Example 1, with the exception that human dermal fibroblasts were not added, in lieu of addition of human dermal fibroblasts at a concentration of 4.0×10⁶ cells/ml.

### Preparation Example 3: Keratinocyte Solution

HEKn cells were harvested using Accutase® (Innovative Cell Technologies, USA) and resuspended in an epidermal cell culture medium to afford a keratinocyte solution having a final cell concentration of 1.2×10⁷ cells/ml.

### Example 1: Artificial Tissue Including Papillae Having Width of 0.3 mm and Height of 50.4 μm

With reference to FIGS. 1 and 2, lyophilized Type-I pig skin extract collagen was dissolved in 0.1 v/v % acetic acid in a dish-shaped plate, added with a nutrient solution containing Dulbecco's Modified Eagle Medium (DMEM), Ham's F-12 and penicillin/streptomycin, uniformly mixed, added with a mixed solution of 0.05 M NaOH, NaHCO₃ and HEPES, and titrated to a neutral pH, thus preparing a mixed solution. Here, the volume ratio of NaOH to NaHCO₃ to HEPES was 8:1:1.

The bioink prepared in Preparation Example 1 was discharged on the mixed solution, thus forming a circular bioink pattern having a size of 0.5 mm.

The mixed solution and the bioink pattern were placed in a CO₂ cell incubator at 37° C. and cured at an elevated temperature. After 2 hr, a cell culture solution was added on the mixed solution and the bioink pattern, the solution was replaced every two days, and the tissue was matured for a predetermined period of time (1-2 weeks), thus manufacturing an artificial tissue including papillae having a width of 0.3 mm and a height of 50.4 μm.

### Example 2: Artificial Tissue Including Papillae Having Width of 0.6 mm and Height of 85.3 μm

An artificial tissue was manufactured in the same manner as in Example 1, with the exception that papillae having a width of 0.6 mm and a height of 85.3 μm were formed by forming a bioink pattern having a size of 1.0 mm, in lieu of formation of the papillae having a width of 0.3 mm and a height of 50.4 μm by forming the bioink pattern having a size of 0.5 mm.

### Example 3: Artificial Tissue Including Papillae Having Width of 1.0 mm and Height of 94.1 Um

An artificial tissue was manufactured in the same manner as in Example 1, with the exception that papillae having a width of 1.0 mm and a height of 94.1 μm were formed by forming a bioink pattern having a size of 2.0 mm, in lieu of formation of the papillae having a width of 0.3 mm and a height of 50.4 μm by forming the bioink pattern having a size of 0.5 mm.

### Example 4: Artificial Skin

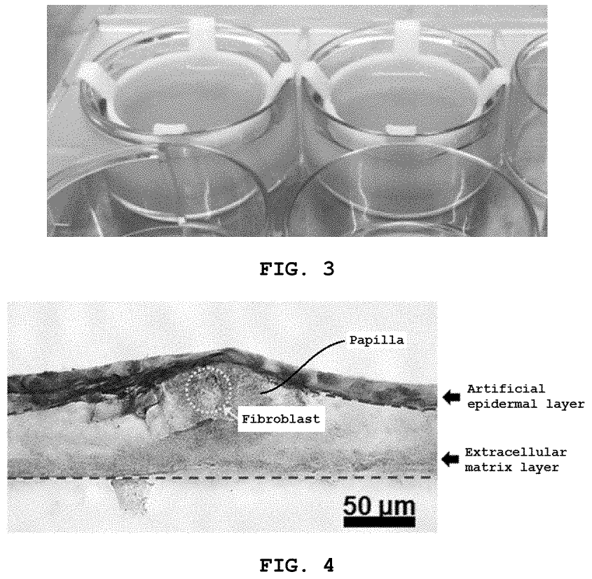

FIG. 3 shows the state in which a keratinocyte solution including keratinocytes is printed and cultured on the cultured mixed solution and bioink pattern in order to manufacture an artificial skin according to an embodiment of the present invention.

With reference to FIGS. 1 to 3, lyophilized Type-I pig skin extract collagen was dissolved in 0.1 v/v % acetic acid in a dish-shaped plate, added with a nutrient solution containing Dulbecco's Modified Eagle Medium (DMEM), Ham's F-12 and penicillin/streptomycin, uniformly mixed, added with a mixed solution of 0.05 M NaOH, NaHCO₃ and HEPES, and titrated to a neutral pH, thus preparing a mixed solution. Here, the volume ratio of NaOH to NaHCO₃ to HEPES was 8:1:1.

The bioink prepared in Preparation Example 1 was discharged on the mixed solution, thus forming a circular bioink pattern having a size of 0.5 mm.

The mixed solution and the bioink pattern were placed in a CO₂ cell incubator at 37° C. and cured at an elevated temperature. After 2 hr, a cell culture solution was added on the mixed solution and the bioink pattern, the solution was replaced every two days, and the tissue was matured for a predetermined period of time (1-2 weeks).

Thereafter, the keratinocyte solution prepared in Preparation Example 3 was printed and cultured on the mixed solution and the bioink pattern, thereby manufacturing an artificial skin.

### Comparative Example 1: Artificial Tissue Manufactured without Discharging Bioink

An artificial tissue was manufactured in the same manner as in Example 1, with the exception that the bioink prepared in Preparation Example 1 was not discharged, in lieu of discharging the bioink prepared in Preparation Example 1 on the mixed solution.

### Comparative Example 2: Artificial Tissue Manufactured by Discharging Bioink not Including Cell

An artificial tissue was manufactured in the same manner as in Example 1, with the exception that the bioink prepared in Preparation Example 2 was used, in lieu of using the bioink prepared in Preparation Example 1.

### Test Examples

### Test Example 1: Cross-Sectional Image of Artificial Skin Tissue

FIG. 4 is an optical microscope image showing the cross-section of the artificial skin manufactured in Example 4. With reference to FIG. 4, it can be confirmed that the artificial papillary layer including papillae was formed on the extracellular matrix layer by contracting and compacting all or part of the collagen due to the traction force of the fibroblast caused by the extracellular matrix receptor (integrin, discoidin domain receptor, glycoprotein VI, immunoglobulin-like cell adhesion molecules, etc.) in the adhesion between the extracellular matrix receptor contained in the fibroblast and the protein and polysaccharide contained in the collagen. Moreover, it can be confirmed that an uneven structure was formed under the epidermis due to the papillae.

### Test Example 2: Bioink Pattern and Papilla Size of Artificial Tissue

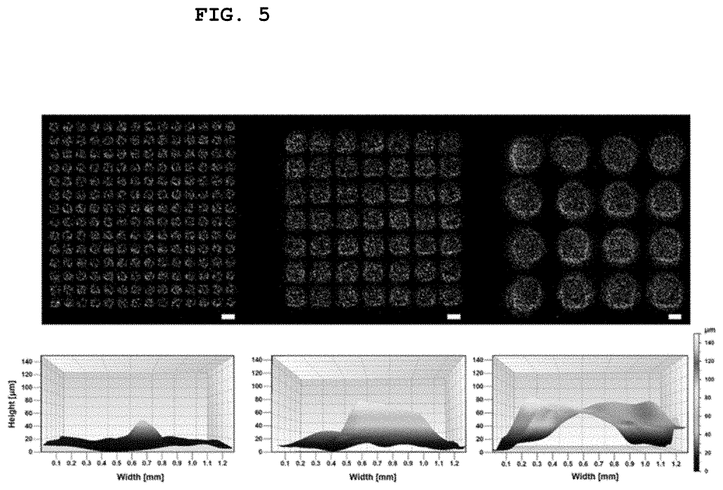

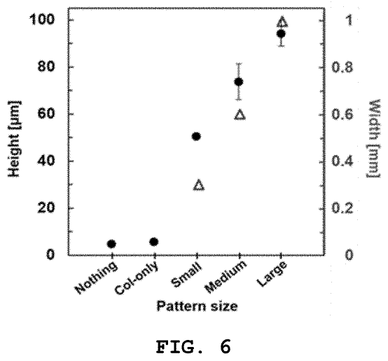

FIG. 5 shows images obtained by scanning the artificial papillary layer of the artificial tissue manufactured in Examples 1 to 3 using a 3D confocal microscope, and FIG. 6 schematically shows the papilla size of the artificial papillary layer in the artificial tissue manufactured in Examples 1 to 3 and Comparative Examples 1 and 2.

With reference to FIGS. 5 and 6, in the artificial tissue of Comparative Example 1, manufactured without discharging the bioink, and in the artificial tissue of Comparative Example 2, manufactured by discharging the bioink not including cells, it can be confirmed that papillae were not formed and thus the artificial papillary layer was not present. On the other hand, in the artificial tissue of Examples 1 to 3 manufactured by discharging the bioink including cells, it can be confirmed that the papillae were formed and thus the artificial papillary layer was present.

Moreover, the height of the formed papillae was increased with an increase in the size of the bioink pattern. When the bioink pattern was formed to a size of 0.5, 1.0 and 2.0 mm in Examples 1, 2 and 3, the formed papillae had a width of 0.3 mm, 0.6 mm and 1.0 mm and a height of 50.4 μm, 85.3 μm and 94.1 lam, respectively.

Therefore, it can be confirmed that the collagen structure was horizontally contracted but vertically raised relative to the size of the printed bioink pattern.

The scope of the invention is defined by the claims below rather than the aforementioned detailed description, and all changes or modified forms that are capable of being derived from the meaning, range, and equivalent concepts of the appended claims should be construed as being included in the scope of the present invention.

What is claimed is:

1. An artificial tissue, comprising:
an extracellular matrix layer comprising a first extracellular matrix; and
an artificial papillary layer formed on the extracellular matrix layer and comprising a papilla or papillae,
each papilla comprising a cell and a second extracellular matrix.

2. The artificial tissue of claim 1, wherein the second extracellular matrix is induced by the cell and contracted.

3. The artificial tissue of claim 1, wherein the cell comprises an extracellular matrix receptor,
the second extracellular matrix comprises at least one selected from the group consisting of a protein and a polysaccharide, and
an adhesion adhering the extracellular matrix receptor to at least one selected from the group consisting of a protein and a polysaccharide is formed.

4. The artificial tissue of claim 3, wherein the adhesion is formed by at least one selected from the group consisting of van der Waals attraction, electrostatic attraction, ionic bonding, hydrogen bonding, and covalent bonding.

5. The artificial tissue of claim 3, wherein the extracellular matrix receptor comprises at least one selected from the group consisting of integrin, discoidin domain receptor, glycoprotein VI, immunoglobulin-like cell adhesion molecule, selectin, syndecan, dystrophin glycoprotein complex, and hyaladherin.

6. The artificial tissue of claim 1, wherein the cell comprises at least one selected from the group consisting of a fibroblast, a myoblast, a nerve cell, and a vascular endothelial cell.

7. The artificial tissue of claim 6, wherein the fibroblast comprises at least one selected from the group consisting of a mammalian fibroblast, an avian fibroblast, a reptile fibroblast, an amphibian fibroblast, and a fish fibroblast.

8. The artificial tissue of claim 1, wherein the artificial papillary layer has a shape corresponding to a pattern.

9. The artificial tissue of claim 8, wherein the pattern comprises at least one selected from the group consisting of a straight-line shape, a curved shape, a polygonal shape, a circular shape, an oval shape, an arc shape, a fan shape and combinations thereof.

10. The artificial tissue of claim 1, wherein each of the extracellular matrix layer and the artificial papillary layer is a hydrogel.

11. The artificial tissue of claim 1, wherein the first extracellular matrix and the second extracellular matrix each independently comprise at least one selected from the group consisting of collagen, gelatin, nanocellulose, fibrinogen, hyaluronic acid, gelatin methacrylate (GelMA), and elastin.

12. An artificial skin, comprising:
an extracellular matrix layer comprising a first extracellular matrix;
an artificial papillary layer formed on the extracellular matrix layer and comprising a papilla or papillae; and
an artificial epidermal layer formed on the artificial papillary layer and comprising a keratinocyte,
each papilla comprising a cell and a second extracellular matrix.

13. The artificial skin of claim 12, wherein the artificial epidermal layer further comprises at least one selected from the group consisting of an epidermal cell and a melanocyte.

14. A method of manufacturing an artificial tissue, comprising:
(a) preparing a mixed solution comprising a first extracellular matrix in a vessel;
(b) forming a bioink pattern by discharging bioink comprising a cell and a second extracellular matrix on the mixed solution; and
(c) culturing the mixed solution and the bioink pattern.

15. The method of claim 14, wherein in step (c), the first extracellular matrix and the second extracellular matrix are each independently cured by at least one selected from the group consisting of van der Waals attraction, hydrogen bonding, ionic bonding, and covalent bonding.

16. The method of claim 15, wherein curing occurs due to a decrease in hydrogen bonding with a water molecule independently of each of the first extracellular matrix and the second extracellular matrix owing to an elevation in temperature, and due to an increase in self-aggregation owing to van der Waals attraction.

17. The method of claim 14, wherein the artificial tissue comprises:
an extracellular matrix layer comprising the first extracellular matrix; and
an artificial papillary layer formed on the extracellular matrix layer and comprising a papilla or papillae,
each papilla comprising the cell and the second extracellular matrix.

18. The method of claim 17, wherein the cell comprises an extracellular matrix receptor,
the second extracellular matrix comprises at least one selected from the group consisting of a protein and a polysaccharide, and
the papilla is formed to protrude by contracting and compacting all or part of the second extracellular matrix due to formation of an adhesion adhering the extracellular matrix receptor to at least one selected from the group consisting of a protein and a polysaccharide.

19. The method of claim 14, wherein the bioink pattern is formed through any one process selected from the group consisting of micro-extrusion printing, inkjet printing, laser printing, valve-type printing, spray printing, micro-stamping, and masking.
